(19) United States
(12) Patent Application Publication
(10) Pub. No.: US 2024/0238041 A1
(43) Pub. Date: Jul. 18, 2024

(54) CATHETER WITH ASYMMETRICAL FIELD DELIVERY

(71) Applicant: Boston Scientific Scimed, Inc., Maple Grove, MN (US)

(72) Inventors: Allan Charles Shuros, St. Paul, MN (US); James Kauphusman, Champlin, MN (US)

(21) Appl. No.: 18/414,133

(22) Filed: Jan. 16, 2024

Related U.S. Application Data
(60) Provisional application No. 63/480,450, filed on Jan. 18, 2023.

(51) Int. Cl. A61B 18/14; A61B 17/00; A61B 18/00; A61B 18/12; A61M 25/00

(52) U.S. Cl. CPC A61B 18/1492; A61B 2017/00526; A61B 2018/00083; A61B 2018/0016; A61B 2018/00267; A61B 2018/00351; A61B 2018/00577; A61B 2018/00613; A61B 2018/00839; A61B 2018/1253; A61M 25/001

(57) ABSTRACT

An electroporation catheter for ablation of cardiac tissue is disclosed. The electroporation catheter includes an elongated, shaft having a distal region and a deflection plane. The elongated shaft defines an axis. An electrode assembly is operably coupled to the distal region and configured to generate an electric field. The electrode assembly includes splines each supporting an ablation electrode. The splines are transitionable between a collapsed configuration and an expanded configuration. A deflector in the distal region is actuatable in the expanded configuration to deflect the electrode assembly off the axis toward a deflection direction in the deflection plane. In the expanded configuration, a first set of splines are proximate the deflection direction and a second set of splines are distal the deflection direction. An insulator is disposed on the second set of splines so as to insulate at least a portion of each associated ablation electrode.

### CROSS REFERENCE TO RELATED APPLICATIONS

The present application claims priority to U.S. Provisional Patent Application Ser. No. 63/480,450, filed Jan. 18, 2023, which is incorporated herein in its entirety.

### TECHNICAL FIELD

The present disclosure relates to medical systems and methods for facilitating ablation of tissue in a patient. More specifically, the present disclosure relates to medical systems and methods for facilitating ablation of tissue by electroporation.

### BACKGROUND

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques can indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electrical field is applied to cells in order to increase the permeability of the cell membrane. The electroporation can be reversible or irreversible, depending on the strength and duration of the electric field. If the electroporation is reversible, the temporarily increased permeability of the cell membrane can be used to introduce chemicals, drugs, or deoxyribonucleic acid (DNA) into the cell, prior to the cell healing and recovering. Tissue recovery can occur over minutes, hours, or days after the ablation is completed. If the electroporation is irreversible, the affected cells are killed, such as via form of cell death, such as perhaps programmed cell death through apoptosis for example, or such as traumatic cell death through necrosis for example.

Irreversible electroporation can be used as a nonthermal ablation technique. In irreversible electroporation, trains of short, high voltage pulses are used to generate electric fields that are strong enough to kill cells. In ablation of cardiac tissue, irreversible electroporation can be a relatively safe and effective alternative to the indiscriminate killing of thermal ablation techniques, such as RF ablation and cryoablation. Irreversible electroporation can be used to kill targeted tissue, such as myocardium tissue, by using a selected electric field strength and duration that is effective to kill the targeted tissue but is not effective to permanently damage other cells or tissue, such as non-targeted myocardium tissue, red blood cells, vascular smooth muscle tissue, endothelium tissue, and nerve cells.

### SUMMARY

In Example 1, an electroporation catheter for ablation of cardiac tissue. The electroporation catheter includes an elongated shaft, an electrode assembly, a deflector, and an insulator. The elongated shaft includes a distal region and a deflection plane, the elongated shaft defines an axis. The electrode assembly is operably coupled to the distal region and configured to generate an electric field. The electrode assembly includes a plurality of splines, and each spline supports an associated ablation electrode. The splines transitionable between a collapsed state and an expanded state. The deflector is in the distal region. The deflector is actuable in the expanded state to deflect the electrode assembly off axis towards a deflection direction in the deflection plane. The electrode assembly in the expanded state includes a first set of the plurality of splines proximal the deflection direction and a second set of the plurality of splines distal the deflection direction. The insulator is disposed on the second set of the plurality of splines so as to insulate at least a portion of the associated ablation electrode.

In Example 2, the electroporation catheter of Example 1, wherein the plurality of splines form a basket defining a cavity in the expanded state, and the insulator directs the electric field from each ablation electrode associated with the second set of the plurality of splines towards the cavity.

In Example 3, the electroporation catheter of Example 2, wherein the basket is steerable toward a treatment site via the deflector.

In Example 4, the electroporation catheter of any of Examples 1-3, wherein the electrode assembly is configured as one of a cathode and an anode to generate the electric field in a monopolar mode.

In Example 5, the electroporation catheter of Example 4, wherein the shaft includes a lead conductor, and each of the associated ablation electrodes are electrically coupled to the lead conductor.

In Example 6, the electroporation catheter of any of Examples 1-5, wherein each ablation electrode associated with the second set of splines include a surface facing the deflection direction and a surface facing opposite the deflection direction, wherein the insulator is disposed on the surface facing opposite the deflection direction.

In Example 7, the electroporation catheter of any of Examples 1-6, wherein each ablation electrode associated with each of the splines include a surface facing the deflection direction and a surface facing the deflection direction, wherein the insulator is disposed on the surface facing opposite the deflection direction.

In Example 8, the electroporation catheter of any of Examples 1-7, wherein each spline is formed of the associated ablation electrode.

In Example 9, the electroporation catheter of any of Examples 1-8 wherein the electrode assembly further includes a mapping electrode.

In Example 10, the electroporation catheter of any of Examples 1-8, wherein each spine further includes a mapping electrode.

In Example 11, the electroporation catheter of Example 10, wherein each spline includes a longitudinal length and wherein each spline includes a plurality of mapping electrodes disposed along the longitudinal length.

In Example 12, the electroporation catheter of any of Examples 9-11, wherein the mapping electrode is not covered by the insulator.

In Example 13, the electroporation catheter of Example 1, wherein each spline includes a resilient support member coupled to the electrode.

In Example 14, the electroporation catheter of Example 13, wherein each spline further includes a plurality of exposed mapping electrodes.

In Example 15, the electroporation catheter of Example 1, wherein each spline supports one associated elongated ablation electrode.

In Example 16, an electroporation catheter for ablation of cardiac tissue. The electroporation catheter includes an elongated shaft, an electrode assembly, a deflector, and an insulator. The elongated shaft includes a distal region and a deflection plane. The elongate shaft defines an axis. The electrode assembly is operably coupled to the distal region and configured to generate an electric field. The electrode assembly includes a plurality of splines, and each spline supports an associated ablation electrode. The splines are transitionable between a collapsed state and an expanded state. The deflector is in the distal region. The deflector is actuable in the expanded state to deflect the electrode assembly off axis towards a deflection direction in the deflection plane. The electrode assembly in the expanded state includes a first set of the plurality of splines proximal the deflection direction and a second set of the plurality of splines distal the deflection direction. The insulator is disposed on the second set of splines so as to insulate at least a portion of the associated ablation electrode.

In Example 17, the electroporation catheter of Example 16, wherein the plurality of splines form a basket defining a cavity in the expanded state, and the insulator directs the electric field from each ablation electrode associated with the second set of the plurality of splines towards the cavity.

In Example 18, the electroporation catheter of Example 17, wherein the basket is steerable toward a treatment site via the deflector.

In Example 19, the electroporation catheter of Example 16, wherein the electrode assembly is configured as one of a cathode and an anode to generate the electric field in a monopolar mode.

In Example 20, the electroporation catheter of Example 19, wherein the shaft includes a lead conductor, and each of the associated ablation electrodes are electrically coupled to the lead conductor.

In Example 21, the electroporation catheter of Example 16, wherein each ablation electrode associated with the second set of splines include a surface facing the deflection direction and a surface facing opposite the deflection direction, wherein the insulator is disposed on the surface facing opposite the deflection direction.

In Example 22, the electroporation catheter of Example 21, wherein each ablation electrode associated with each of the splines include a surface facing the deflection direction and a surface facing the deflection direction, wherein the insulator is disposed on the surface facing opposite the deflection direction.

In Example 23, the electroporation catheter of Example 16, wherein ablation electrodes associated with the first set of the plurality of splines are exposed.

In Example 24, the electroporation catheter of Example 16, wherein each spine further includes a mapping electrode.

In Example 25, the electroporation catheter of Example 24, wherein each spline includes a plurality of exposed mapping electrodes disposed along the spline.

In Example 26, an electroporation catheter for ablation of cardiac tissue. the electroporation catheter including an elongated shaft, an electrode assembly, a deflector, and an insulator. The elongated shaft includes a distal region and a deflection plane. The elongated shaft defines an axis. The electrode assembly is operably coupled to the distal region and configured to generate an electric field. The electrode assembly includes a plurality of elongated ablation electrodes configured as splines. Each ablation electrode includes an outer conductive surface and an inner conductive surface, and each spline supports a mapping electrode. The splines are transitionable between a collapsed state and an expanded state. The deflector is in the distal region. The deflector is actuable in the expanded state to deflect the electrode assembly off axis towards a deflection direction in the deflection plane. The electrode assembly in the expanded state includes a first set of the plurality of splines proximal the deflection direction and a second set of the plurality of splines distal the deflection direction. The insulator is disposed on the second set of the plurality of splines so as to insulate at least a portion of the outer conductive surface and expose the mapping electrode.

In Example 27, the electroporation catheter of Example 26, wherein the plurality of splines form a basket defining a cavity in the expanded state, and wherein each inner conductive surface faces towards the cavity.

In Example 28, the electroporation catheter of Example 27, wherein the insulator disposed on the second set of the plurality of splines does not insulate the inner conductive surface.

In Example 29, the electroporation catheter of Example 28, wherein the insulator is further disposed on the first set of splines so as to insulate the inner conductive surface and not insulate the outer conductive surface of the first set of splines.

In Example 30, the electroporation catheter of Example 26, wherein the shaft includes a lead conductor, and each of the ablation electrodes are electrically coupled to the lead conductor.

In Example 31, a method for making an electroporation catheter for ablation of cardiac tissue. An elongated shaft having a distal region and a deflection plane is provided. The elongate shaft defines an axis. An electrode assembly is coupled to the distal region. The electrode assembly is configured to generate an electric field. The electrode assembly includes a plurality of splines, and each spline supports an associated ablation electrode. The splines are transitionable between a collapsed state and an expanded state. A deflector is provided in the distal region. The deflector is actuable in the expanded state to deflect the electrode assembly off axis towards a deflection direction in the deflection plane. The electrode assembly in the expanded state includes a first set of the plurality of splines proximal the deflection direction and a second set of the plurality of splines distal the deflection direction. An insulator is deposited on the second set of the plurality of splines so as to insulate at least a portion of the associated electrode.

In Example 32, the method of Example 31, wherein providing the shaft includes providing a lead conductor in the shaft, and further comprising electrically coupling each of the associated ablation electrodes to the lead conductor.

In Example 33, the method of Example 31, and further including each of the splines are formed from the associated ablation electrodes.

In Example 34, the method of Example 33, and further each of the splines are formed to create a basket in the expanded state.

In Example 35, the method of Example 31, and further including a plurality of mapping electrodes are attached to each spline, wherein the insulator is not deposited on the mapping electrodes.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system, in accordance with embodiments of the subject matter of the disclosure.

FIG. 2 is a schematic diagram illustrating an example catheter that can be used with the example electrophysiology system of FIG. 1.

FIG. 3A is a sectioned front schematic view of a first example electrode assembly that can be used with the example catheter of FIG. 2.

FIG. 3B is a sectioned front schematic view of a second example electrode assembly that can be used with the example catheter of FIG. 2.

FIG. 3C is a sectioned front schematic view of a third example electrode assembly that can be used with the example catheter of FIG. 2.

FIG. 3D is a sectioned front schematic view of a fourth example electrode assembly that can be used with the example catheter of FIG. 2.

FIG. 3E is a sectioned front schematic view of a fifth example electrode assembly that can be used with the example catheter of FIG. 2.

FIG. 4 is a top view of a portion of the fourth example electrode assembly of FIG. 3D.

FIG. 5 is a perspective illustration of a distal portion of an example splined catheter for use in the electrophysiology system of FIG. 1, in accordance with the example catheter of FIG. 2.

FIG. 6 is a sectioned front schematic view of a portion of the electrode assembly of the example splined catheter of FIG. 5.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) of the features in an example used across all examples. Thus, no one figure should be interpreted as having any dependency or requirement related to any single component or combination of components illustrated therein. Additionally, various components depicted in a figure may be, in examples, integrated with various ones of the other components depicted therein (or components not illustrated), all of which are considered to be within the ambit of the present disclosure.

As applied to electrophysiology systems, irreversible electroporation uses high voltage, short pulses to kill cells such as myocardium through apoptosis while sparing other adjacent tissues including the esophageal vascular smooth muscle and endothelium. Irreversible electroporation treatment can be delivered in multiple therapy sections. A therapy section, which may have a duration on the order of milliseconds, may include a plurality of electrical pulses, such as a few dozen pulses, generated and delivered by an electroporation device, which is powered by an electroporation generator, to generate an electric field of sufficient strength to create transmural lesions. Such pulsed field ablation creates generally spherical electric fields around a generally point electrode, or electric fields having generally circular cross sections around a generally linear electrode, that is generally independent of tissue or blood in the heart. Such fields can include the potential to cause collateral damage to adjacent areas on the blood pools side of the electrode, or side opposite to the target tissue. In contrast, RF ablation can be focused to areas in direct contact with the electrode.

Electroporation devices that perform ablation and cardiac mapping, such as mapping and ablation catheters, generally are of a larger size than many RF ablation catheters. For example, a working end of a mapping and ablation catheter may be 7 mm to 15 mm or more in diameter. Such catheters often produce relatively large electric fields for ablation, which increase the potential for collateral damage to areas opposite the myocardial target tissue.

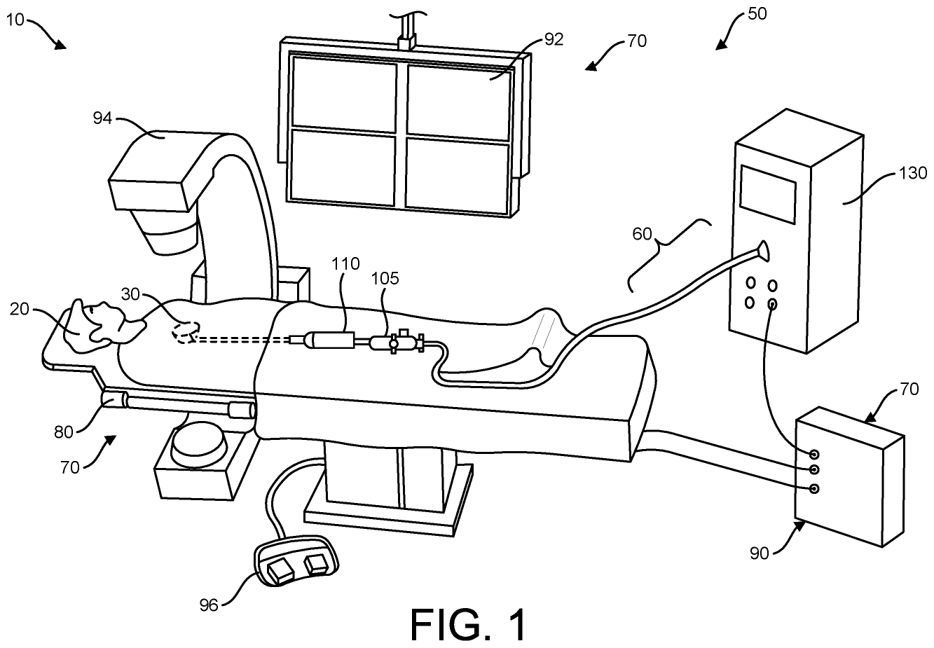

FIG. 1 illustrates an example clinical setting 10 for treating a patient 20, such as for treating a heart 30 of the patient 20, using an electrophysiology system 50, in accordance with the disclosure. The electrophysiology system 50 includes an electroporation catheter system 60 and an electro-anatomical mapping (EAM) system 70. The example electroporation catheter system 60 includes an electroporation catheter 105, an introducer sheath 110, and an electroporation console 130. Additionally, the electroporation catheter system 60 includes various connecting elements, such as cables, that operably connect the components of the electroporation catheter system 60 to one another and to the components of the EAM system 70. In general, the EAM mapping system 70 includes a localization field generator 80, a mapping and navigation controller 90, and a display 92. Also, the clinical setting 10 can include additional equipment such as imaging equipment 94 (represented by the C-arm) and various controller elements, such as a foot controller 96, configured to allow an operator to control various aspects of the electrophysiology system 50. The clinical setting 10 may have other components and arrangements of components that are not shown in FIG. 1.

The electroporation catheter system 60 is configured to deliver ablation electric field energy to targeted tissue in the patient's heart 30 to create cell death in tissue, for example, rendering the tissue incapable of conducting electrical signals. Also, the electroporation catheter system 60 is configured to generate electric fields using the electroporation catheter 105 to create and present, on the display 92, an electro-anatomical map of the patient's heart to aid a clinician in planning ablation by irreversible electroporation using the electroporation catheter 105 prior to delivering ablation electric field energy. In embodiments, the electroporation catheter system 60 is configured to generate the electric fields based on characteristics of the electroporation catheter 105 and the position of the electroporation catheter 105 in the patient 20, such as in the heart 30 of the patient 20. The electroporation catheter system 60 is configured to generate graphical representations of the electroporation catheter and the electro-anatomical map based on characteristics of the electroporation catheter 105 and the position of the electroporation catheter 105 in the patient 20, such as in the heart 30 of the patient 20, and the characteristics of the tissue surrounding the catheter 105, such as measured impedances of the tissue. In one example, the electroporation catheter 105 is a mapping and ablation catheter, which can be deployed in mapping procedures in cooperation with the EAM system 70 as well as to deliver ablation electric field energy and ablate tissue via irreversible electroporation.

The introducer sheath 110 is operable to provide a delivery conduit through which the electroporation catheter 105 can be deployed to the specific target sites within the patient's heart 30. Access to the patient's heart can be obtained through a vessel, such as a peripheral artery or vein. Once access to the vessel is obtained, the electroporation catheter 105 can be navigated to within the patient's heart, such as within a chamber of the heart.

The example electroporation catheter 105 includes an elongated catheter shaft and distal end region configured to be deployed proximate target tissue, such as within a chamber of the patient's heart. The shaft can extend from an access point in the patient to the target tissue and generally defines a longitudinal axis of the electroporation catheter 105. The distal end region may include a basket, balloon, spline, configured tip, or other electrode deployment mechanism coupled to the shaft. The electrode deployment mechanism includes an electrode assembly, or array, comprising an electrode. For example, the electrode assembly can include a plurality of spaced-apart electrodes or multiple spaced-apart sets or groups of spaced-apart electrodes. In some examples, an electrode, such as a plurality of spaced-apart electrodes, can be deployed on the catheter shaft in addition to or instead of an electrode on the electrode deployment mechanism. For instance, the electrode deployment mechanism includes a plurality of splines configured to form a basket, and at least a some of the electrodes are disposed on the splines.

The electroporation catheter 105 is configurable in a plurality of configurations. For example, when the distal end region of the catheter 105 is within a sheath as a catheter assembly, such as to travel to the patient to the chamber of the heart, the electrode deployment mechanism and electrode assembly are in a collapsed state to fit within the sheath. Once the catheter has reached the destination in the chamber of the heart, for example, the sheath is retracted from the distal region of the catheter 105 (or the shaft catheter is extended past the sheath) and the electrode deployment mechanism and electrode assembly can be arranged in an expanded state. The electrode assembly has a collapsed shape when the catheter 105 is in the collapsed state and an expanded shape when the catheter 105 is in the expanded state. In some examples, the electrode assembly has more than two states.

In one example, the plurality of electrodes can be formed of a conductive, solid-surface, biocompatible material and are spaced-apart across insulators. Each of the plurality of electrodes is electrically coupled to a corresponding elongated lead conductor that extend along the shaft to a catheter proximal end. In one example, each electrode of the spaced-apart electrodes corresponds with a separate, single lead conductor. In another example, a plurality of electrodes may be coupled to a single lead conductor. Other configurations are contemplated. The plurality of lead conductors can be insulated from one another within an insulating sheath along the catheter shaft, such as with an insulating polymer sheath. The lead conductors can be electrically coupled to plug in the proximal region of the electroporation catheter 105, such as a plug configured to be mechanically and electrically coupled to the electroporation console 130, for example, either directly or via intermediary electrical conductors such as cabling.

The electroporation console 130 includes a controller, such one or more controllers, processors, or computers, that executes instructions or code, such as processor-executable instructions, out of a non-transitory computer readable medium, such as a memory device, or memory, to cause, such as control or perform, the aspects of the electroporation catheter system 60. In one example, the electroporation console 130 is configured to provide an electrical signal, such as a plurality of concurrent or space-apart-time electrical signals, to the electrically connected electroporation catheter 105 along lead conductors to the spaced-apart electrodes. The spaced-apart electrodes are configured to generate a selected electrical field proximate the target tissue, based on the electrical signals from the electroporation console 130, such as to effect ablation.

The electroporation console 130 can generate electrical signals and select which electrodes in the electrode array will receive the electrical signals. A first electrode, or first group of electrodes, can be selected to be an anode and a different, second electrode, or second group of electrodes, can be selected to be a cathode, such that electrical fields can be generated between the anode and cathode based on signals, such as pulses, provided to the electrodes from the electroporation console 130. The console 130 provides electric pulses of different lengths and magnitudes to the electrodes on the catheter 105. The electric pulses can be provided in a continuous stream of pulses or in multiple, separate trains of pulses. Pulse parameters of interest include the number of pulses, the duty cycle of the pulses, the spacing of pulse trains, the voltage or magnitude of the pulses including the peak voltages, and the duration of the voltages. For example, the console 130 can select two or more electrodes of the electrode assembly and provides pulses to the selected electrodes to generate electric fields between the selected electrodes.

In an ablation mode, the console can select electrodes to provide pulsed field ablation (PFA). For example, PFA can be performed with monophasic waveforms and biphasic waveforms. Without being bound to a particular theory, electric field strengths in the range of generally 200-250 volts per centimeter (V/cm) with microsecond-scale pulse duration have been demonstrated to provide reversible electroporation in cardiac tissue. Electric field strengths at approximately 400 V/cm have been demonstrated to provide irreversible electroporation in cardiac tissue of interest, such as targeted myocardium tissue and endocardium tissue, with demonstrable sparing of red blood cells, vascular smooth muscle tissue, endothelium tissue, nerves and other non-targeted proximate tissue.

Additionally, the electrode assembly on catheter 105 can be operated in a selected mode such as monopolar mode or bipolar mode. During monopolar operation of the catheter 105, an electrode, a group of electrodes, or the entire electrode assembly are configured as one of an anode or a cathode. None of the electrodes in the electrode assembly are configured as a the other of the cathode or the anode. Instead, the other of the cathode or the anode is provided in the form of a pad dispersive electrode located on the patient, typically on the back, buttocks, or other suitable anatomical location during electroporation. An electrical field is formed between an activated electrode of the electrode assembly and the pad dispersive electrode. In an alternative configuration, a return electrode, such as a plurality of return electrodes, can be disposed on the shaft of the catheter. An electrical field is formed between an activated electrode of the electrode assembly and the return electrode on the shaft of the catheter 105. During bipolar operation of the catheter 105, a first set of one or more electrodes of the electrode assembly, is configured as the anode and a second set of one or more electrodes of the electrode assembly, is configured as the cathode, to generate the electric field. In this example, a pad dispersive electrode is not used, and the electrical field is not extended in the patient's body, but rather through a localized portion of tissue proximate the electrode assembly.

In some examples, the catheter 105 can be a mapping and ablation catheter, and the electrodes can include ablation electrodes that are configured to deliver ablation electric field energy and mapping electrodes for mapping purposes. In some configurations, the mapping electrodes are configured to be used to collect electrical signals to be used to generate via the operably coupled EAM system 70, and display via the operably coupled display 92, detailed three-dimensional geometric anatomical maps or representations of the cardiac chambers as well as electro-anatomical maps in which cardiac electrical activity of interest is superimposed on the geometric anatomical maps. In some examples, an electrode can operate as an ablation electrode in an ablation mode of the electrophysiology system 50 and as a mapping electrode in a mapping mode of the system 50. Mapping electrodes on the electroporation catheter 105 can measure electrical signals and generate output signals that can be processed by the mapping and navigation controller 90 to generate an electro-anatomical map. In some instances, electro-anatomical maps are generated before ablation for determining the electrical activity of the cardiac tissue within a chamber of interest. In some instances, electro-anatomical maps are generated after ablation in verifying the desired change in electrical activity of the ablated tissue and the chamber. The mapping electrodes may also be used to determine the position of the catheter 105 in three-dimensional space within the body. For example, when the operator moves the distal end of the catheter 105 within a cardiac chamber of interest, the boundaries of catheter movement can be used by the mapping and navigation controller 90 to form the anatomical map of the chamber. The chamber anatomical map may be used to facilitate navigation of the catheter 105 without the use of ionizing radiation such as with fluoroscopy, and for tagging locations of ablations as they are completed in order to guide spacing of ablations and aid the clinician in ablating the anatomy of interest. In some examples, an electrode in the electrode assembly can be configured to only perform an ablation or the electrode in the electrode assembly can be configured to only perform mapping.

The EAM system 70 is configured to generate the electro-anatomical map for display on the display 92. The EAM system 70 is operable to track the location of the various components of the electroporation catheter system 60, and to generate high-fidelity three-dimensional anatomical and electro-anatomical maps of the heart, including portions of the heart such as cardiac chambers of interest or other structures of interest such as the sinoatrial node or atrioventricular node. In one illustrative example, the EAM system 70 can include the RHYTHMIA™ HDx mapping system marketed by Boston Scientific Corporation. Also, the mapping and navigation controller 90 of the EAM system 70 includes one or more controllers, such as microprocessors or computers, that execute code out of memory to control or perform functional aspects of the EAM system 70, in which the memory, can be part of the one or more controllers, microprocessors, computers, or part of a memory device accessible through a computer network.

The EAM system 70 generates a localization field, via the field generator 80, to define a localization volume about the heart 30, and a location sensor or sensing element on a tracked device, such as sensors on the electroporation catheter 105, generate an output that can be processed by the mapping and navigation controller 90 to track the location of the sensor, and consequently, the corresponding device, within the localization volume. In the illustrated example, the device tracking is accomplished using magnetic tracking techniques, in which the field generator 80 is a magnetic field generator that generates a magnetic field defining the localization volume, and location sensors on the tracked devices are magnetic field sensors.

In other examples, impedance tracking methodologies may be employed to track the locations of the various devices. In such examples, the localization field is an electric field generated, for example, by an external field generator arrangement, such as surface electrodes, by intra-body or intra-cardiac devices, such as an intracardiac catheter, or both. In these examples, the location sensing elements can constitute electrodes on the tracked devices that generate outputs received and processed by the mapping and navigation controller 90 to track the location of the various location sensing electrodes within the localization volume.

The EAM system 70 can be equipped for both magnetic and impedance tracking capabilities. In such examples, impedance tracking accuracy can, in some instances be enhanced by first creating a map of the electric field induced by the electric field generator within the cardiac chamber of interest using a probe equipped with a magnetic location sensor, as is possible using the RHYTHMIA HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the electroporation catheter 105 or another catheter or probe equipped with sensing electrodes, to generate, and display via the display 92, detailed three-dimensional geometric anatomical maps or representations of the heart tissue and voids such as cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map or the electro-anatomical map.

The electroporation catheter system 60 can be combined or integrated with the EAM system 70 to allow graphical representations of the electric fields that can be produced by the electroporation catheter 105 to be visualized on an electro-anatomical map of the patient's heart. The integrated system can include the capability to enhance the efficiency of clinical workflows, including enhancement of providing a visual representation to the clinician of ablation lesions of portions of the patient's heart created through irreversible electroporation. The integrated system can include generating the graphical representations of the electric fields that can be produced by the electroporation catheter 105, generating the anatomical maps including generating the electro-anatomical maps, and displaying information related to the location and electric field strengths of the electric fields that can be produced by the electroporation catheter 105.

The depiction of the electrophysiology system 50 shown in FIG. 1 is intended for illustration or a general overview of the various components of the system 50 and is not intended to imply that the disclosure is limited to any set of components or arrangement of the components. For example, additional hardware components, such as breakout boxes or workstations, can be included in the electrophysiology system 50.

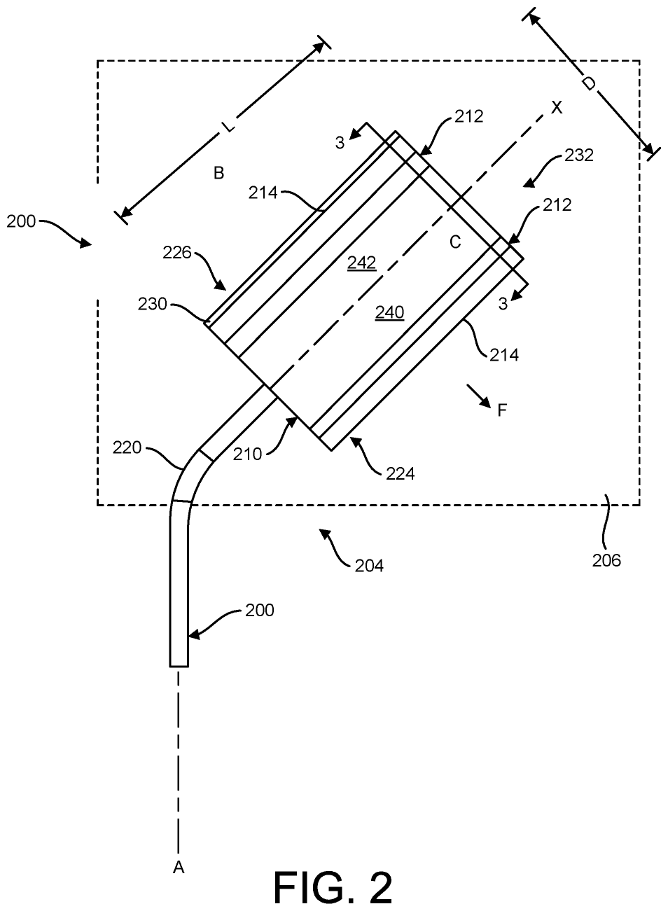

FIG. 2 illustrates an example electroporation catheter 200 for ablation of cardiac tissue, which can be an example of catheter 105 and used with the electrophysiology system 50. The example electroporation catheter 200 includes an elongated shaft 202 having a distal region 204 and a deflection plane 206. The distal region 204 is configured to be deployed proximate target tissue, such as within a chamber of the patient's heart. The elongate shaft 202 defines a longitudinal axis A. The longitudinal axis A is presented as a line passing through a centroid of a cross section of the shaft 202. An electrode assembly 210 is operably coupled to the distal region 204 and configured to generate an electric field. In one example, the electric field effects an irreversible electroporation in selected cardiac tissue. The electrode assembly 210 includes a plurality of splines 212, and each spline of the plurality of spline 212 supports an associated electrode 214. In one example, each spline supports an associated ablation electrode. For instance, each spline supports a plurality of associated ablation electrodes. The splines 212 are transitionable between a collapsed state and an expanded state. A deflector 220 is in the distal region 204. The deflector 220 is actuable in the expanded state to deflect the electrode assembly 210 off axis A toward a deflection direction F in the deflection plane 206. For example, the deflector 220 is coupled to the shaft 202, and the axis A is determined proximal the deflector 220. The electrode assembly 210 in the expanded state includes a first set 224 of the plurality of splines 212 proximal the deflection direction F and a second set 226 of the plurality of splines 212 distal the deflection direction F. For example, the first set 224 can include one or more splines, and the second set 226 can include one or more splines. An insulator 230 is disposed on the second set 226 of the plurality of splines 212 so as to insulate at least a portion of the associated electrode 214. For example, the insulator 230 is disposed on the second set 226 of the plurality of splines 212 on a side opposite the deflection direction F. The insulator 230 not is disposed on the associated electrodes 214 of the first set 224 of the plurality of electrodes.

In the illustrated example, the splines 212 form a basket 232 coupled to the shaft 202. The basket 232 in the expanded state can include an expanded length L and an expanded diameter D, and a particular contour. The length of the basket 232, such as length L, can be determined from the longitudinal distance of the spines 212 from a proximal end to a distal end along a basket axis X. The diameter of the basket 232, such as diameter D, can be determined from the greatest distance between opposite splines 212 in a direction generally perpendicular to the basket axis X. In the expanded state, the plurality of splines 212 are spaced-apart from each other and form a cavity C within the basket 232. In the example, the plurality of spline 212 are constructed from a flexible and resilient material such that the catheter 200 can also be transitionable to a collapsed state (not shown) in which the collapse length of the basket 232, in this example, is greater than L and the collapsed diameter of the basket is less than D. In one example, the catheter 200 is placed in the collapsed state while a deployment sheath is disposed over the splines 212, and the catheter 200 is in a non-operational mode. If the basket 232 is extended from the deployment sheath, or the deployment sheath is retracted from the basket 232, the basket can assume the expanded state and be deployed in an operational mode.

The deflector 220 permits the basket 232 to deflect, or move, off axis A, such as toward a treatment site in the deflection plane 206. In one example, the deflector 220 allows the basket 232 to pivot or bend in the deflection plane 206 relative to a region on the shaft proximal to the deflector 220. For illustration, the deflector 220 can allow the basket to pivot about a pivot axis generally perpendicular to the deflector plane 206. As the basket 232 moves off axis, the basket 232 moves in a deflection direction F, such as toward the treatment site. As the deflected basket moves toward the axis A, the basket returns to the axis A in an opposite direction B. When the basket 232 in on axis, axis A and basket axis X are parallel or colinear. For instance, the shaft 202 can include pull wires (not shown) mechanically coupled, such as via a ring around the shaft, to the distal region 204 and mechanically coupled to a proximal end of the catheter, such as to a handle, to permit a clinician to actuate movement of the basket 232 off axis. Tension is applied to the pull wires to deflect the electrode assembly off axis toward the deflection direction F in the deflection plane and to selectively steer the electrode assembly toward a treatment site.

The electrode assembly 210 in the expanded state includes the first set 224 of the plurality of splines 212 proximal the deflection direction F and the second set 226 of the plurality of splines 212 distal the deflection direction F. In one example, the basket axis X lies in bisector plane perpendicular to the deflection plane 206. The first set 224 of the plurality of splines 212 are on a proximal side 240 of the bisector plane (indicated in FIG. 2 as including basket axis X) proximal the deflection direction F. The second set 226 of the plurality of splines 212 are on a distal side 242 of the bisector plane distal the deflection direction F. The insulator 230 is disposed on the second set 226 of the plurality of splines 212 so as to insulate the associated electrode 214. For example, the insulator 230 is disposed on ablation electrodes on the second set 226 of the plurality of splines 212 on a side opposite the deflection direction F and facing away from cavity C. The insulator 230 is not disposed on electrode surfaces facing toward the cavity C. For electrodes 214 associated with the second set 226 of the plurality splines 212, electrode surfaces of configured to face toward the cavity C are exposed. Also, the insulator is not disposed on mapping electrodes, and the mapping electrodes are exposed. For ablation electrodes 214 associated with the second set 226 of the plurality splines 212, electrode surfaces of configured to face away from the cavity C are insulated. Further, the insulator 230 not is disposed on the associated electrodes 214 of the first set 224 of the plurality of electrodes.

In one example, the catheter 200 is be operated in a monopolar mode to generate an electric field for electroporation. For example, the ablation electrodes on the first set 224 and second set 226 of splines 212 can be electrically coupled to a single lead conductor that extends the length of the shaft 202, or to a set of lead conductors in the shaft 202 that are configured to carry the same electrical signal to generate the electric field. The ablation electrodes on the electrode assembly 210 are configured as one of the anode or the cathode. None of the electrodes in the electrode assembly are configured as the other of the cathode or the anode. Instead, the other of the cathode or the anode is provided in the form of a pad dispersive electrode located on the patient during electroporation. The electrical field is formed between the activated electrodes of the electrode assembly and the pad dispersive electrode. Via the application of the insulator 230 on the second set 226 of splines 212, the electrode assembly 210 projects an asymmetrical electric field that focuses electroporation energy into the cavity C and toward to the deflection direction F rather than symmetrically in all directions radially from the basket axis X. Lateral electric field projection corresponds with the deflection plane 206, and the electric field is steered toward the treatment tissue.

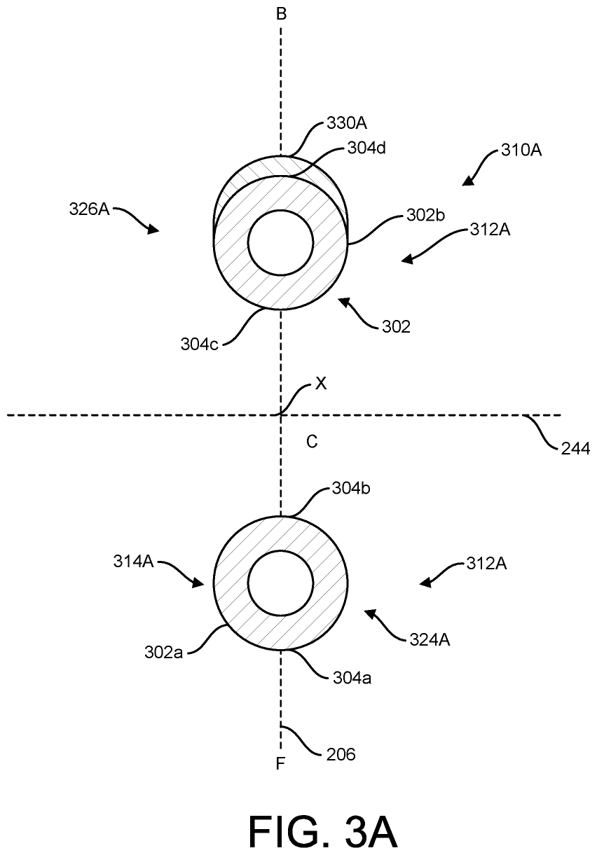

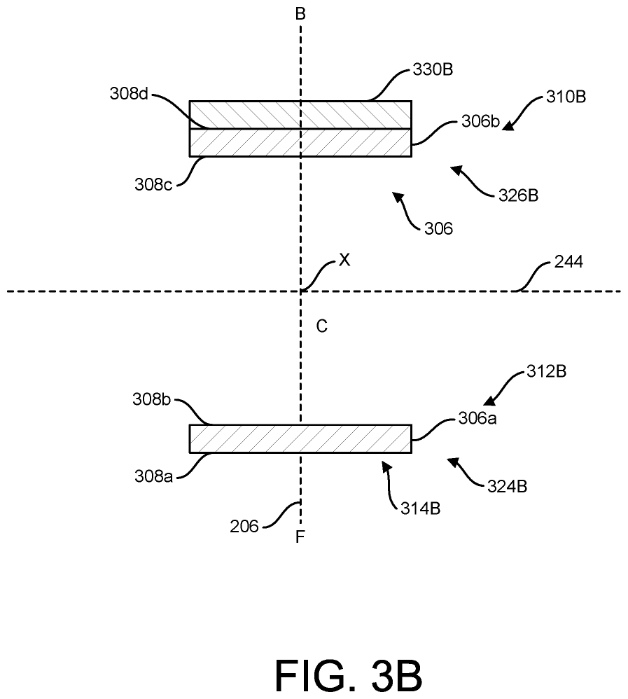

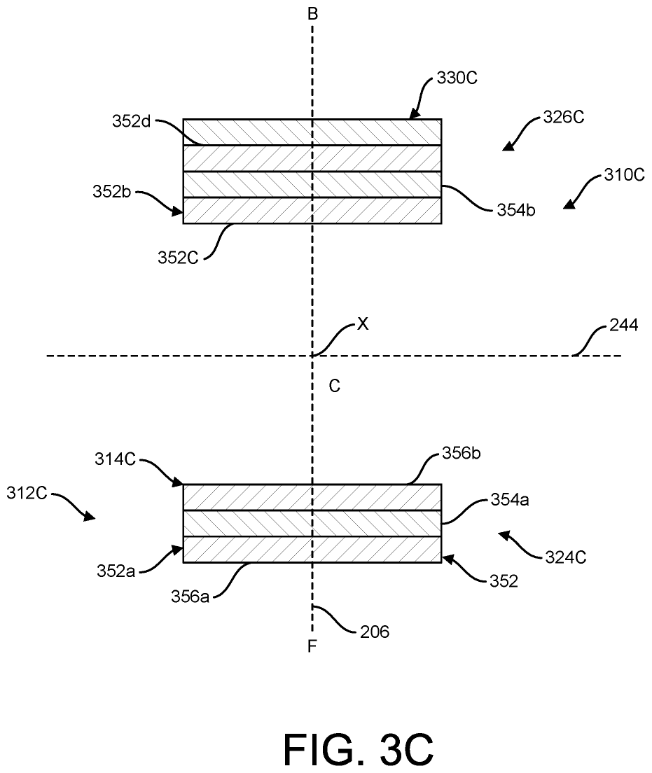

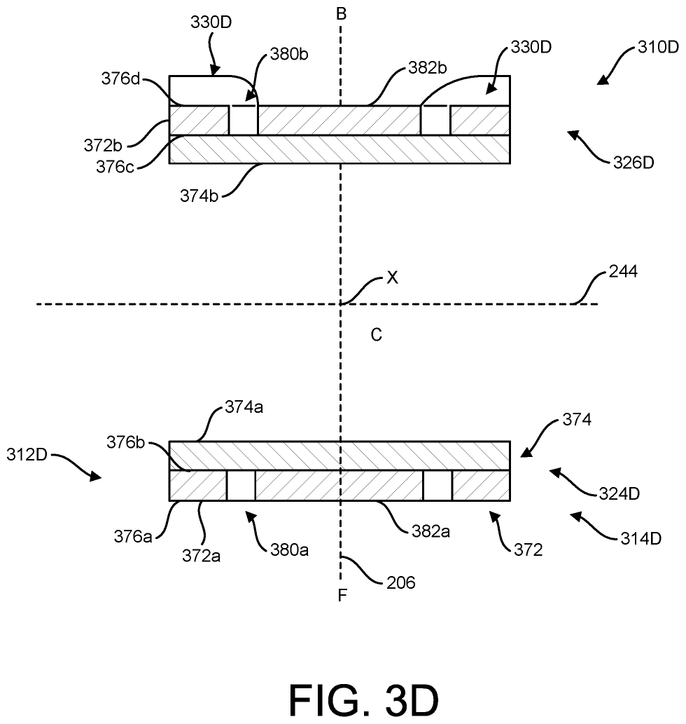

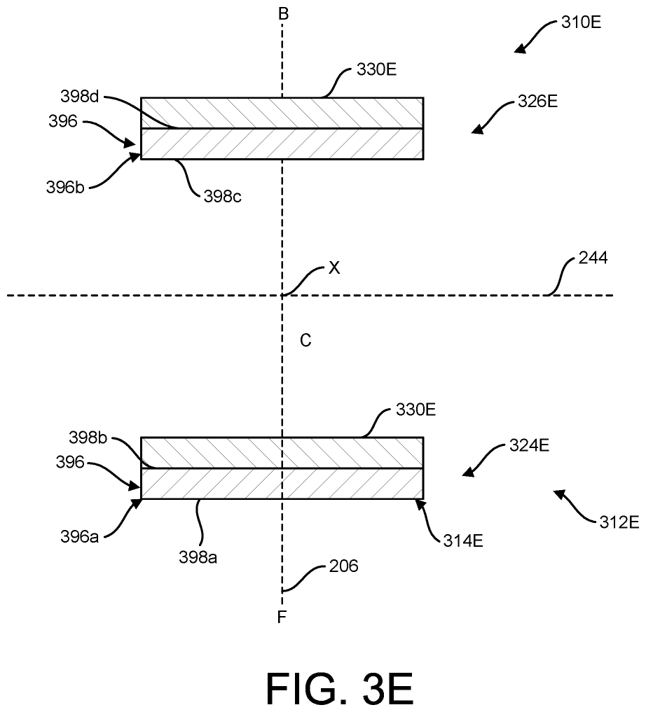

FIGS. 3A-3E illustrate several examples of many examples in accordance with the features of the catheter 200. The examples of FIGS. 3A-3E are presented as schematic illustrations of cross sections that can be representations of the catheter 200 as viewed along lines 3-3 in FIG. 2. For instance, FIGS. 3A-3E illustrate the deflection plane 206, basket axis X in the deflection plane 206, bisector plane 244 as including basket axis X and perpendicular to deflection plane 206, deflection direction F, cavity C, and opposite direction B.

FIG. 3A illustrates an example electrode assembly 310A in the expanded state and includes a first set 324A of a plurality of splines 312A proximal the deflection direction F and a second set 326A of the plurality of splines 312A distal the deflection direction F. One spline from each set 324A, 326A is illustrated in the example, but the first set 324A can include one or more splines, and the second set 326A can include one or more splines. In the example, a ring electrode 302 is associated with each spline 312A. For instance, each spine 312A can include a plurality of longitudinally spaced-apart ring electrodes, from which at least one from each spline can be used for ablation, such as ring electrode 302a associated with the first set spine 324A and ring electrode 302b associated with the second set spline 326A. The ring electrode 302a associated with the first set spine 324A includes a portion of the surface 304a facing the deflection direction F and a portion of the surface 304b facing the cavity C. The ring electrode 302b associated with second set spline 326A includes a portion of the surface 304c facing the cavity C and a portion of the surface 304d facing opposite direction B. An insulator 330A is disposed on the second set 326A of the plurality of splines 312A so as to insulate the associated ring electrode 302b. For example, the insulator 330A is disposed on the second set 326A of the plurality of splines 312A on a side opposite the deflection direction F, which provides for the insulator 330A on surface 304d of the ring electrode 302b. The insulator 330A is not is disposed on the associated electrodes 314A of the first set 324A of the plurality of electrodes. Additionally, the insulator 330A is not disposed on electrode surfaces facing toward the cavity C, such as surface 304c of ring electrode 302b.

FIG. 3B illustrates an example electrode assembly 310B in the expanded state and includes a first set 324B of a plurality of splines 312B proximal the deflection direction F and a second set 326B of the plurality of splines 312B distal the deflection direction F. One spline from each set 324B, 326B is illustrated in the example, but the first set 324B can include one or more splines, and the second set 326B can include one or more splines. In the example, a plate electrode 306 is associated with each spline 312B. For instance, each spine 312B is formed from a longitudinally extending plate electrode, which can be used for ablation, such as plate electrode 306a associated with the first set spine 324B and plate electrode 306b associated with the second set spline 326B. The plate electrode 306a associated with the first set spine 324B includes a surface 308a facing the deflection direction F and a surface 308b facing the cavity C. The plate electrode 306b associated with second set spline 326B includes a surface 308c facing the cavity C and a surface 308d facing opposite direction B. An insulator 330B is disposed on the second set 326B of the plurality of splines 312B so as to insulate the associated plate electrode 306b. For example, the insulator 330B is disposed on the second set 326B of the plurality of splines 312B on a side opposite the deflection direction F, which provides for the insulator 330B on surface 308d of the plate electrode 306b. The insulator 330B is not is disposed on the associated electrodes 314B of the first set 324B of the plurality of electrodes. Additionally, the insulator 330B is not disposed on electrode surfaces facing toward the cavity C, such as surface 308c of plate electrode 308b.

FIG. 3C illustrates an example electrode assembly 310C in the expanded state and includes a first set 324C of a plurality of splines 312C proximal the deflection direction F and a second set 326C of the plurality of splines 312C distal the deflection direction F. One spline from each set 324C, 326C is illustrated in the example, but the first set 324C can include one or more splines, and the second set 326C can include one or more splines. In the example, a plate electrode 352 is associated with each spline 312C. For instance, each spine 312C can include a resilient support member 354 upon which one plate electrode or a plurality of longitudinally spaced-apart plate electrodes, from which at least one from each spline can be used for ablation, such as plate electrode 352a associated with the first set spine 324C and plate electrode 352b associated with the second set spline 326C. The plate electrode 352a associated with the first set spine 324C includes a surface 356a facing the deflection direction F and a surface 356b facing cavity C that is coupled to support member 354a. The plate electrode 352b associated with second set spline 326C includes a surface 356c facing cavity C that is coupled to support member 354b and a surface 356d facing opposite direction B. An insulator 330C is disposed on the second set 326C of the plurality of splines 312C so as to insulate the associated plate electrode 352b. For example, the insulator 330C is disposed on the second set 326C of the plurality of splines 312C on a side opposite the deflection direction F, which provides for the insulator 330C on surface 356d of the plate electrode 352b. The insulator 330C is not is disposed on the associated electrodes 314C of the first set 324C of the plurality of electrodes.

FIG. 3D illustrates an example electrode assembly 310D in the expanded state and includes a first set 324D of a plurality of splines 312D proximal the deflection direction F and a second set 326D of the plurality of splines 312D distal the deflection direction F. One spline from each set 324D, 326D is illustrated in the example, but the first set 324D can include one or more splines, and the second set 326D can include one or more splines. In the example, a plate electrode 372 is associated with each spline 312D. For instance, each spine 312D can include a resilient support member 374 upon which one plate electrode or a plurality of longitudinally spaced-apart plate electrodes, from which at least one from each spline can be used for ablation, such as plate electrode 372a associated with the first set spine 324D and plate electrode 372b associated with the second set spline 326D. The plate electrode 372a is disposed on support member 374a. The plate electrode 372a includes an aperture 380a, and a mapping electrode 382a is disposed onto the first support member 374a. The plate electrode 372b is disposed on support member 374b. The plate electrode 372b includes an aperture 380b, and a mapping electrode 382b is disposed onto the first support member 374b.

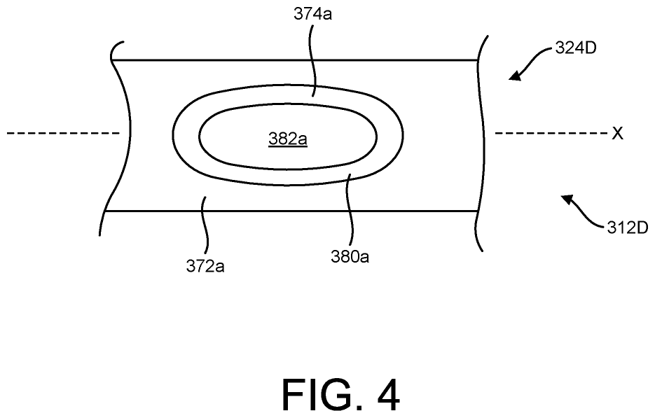

FIG. 4 illustrates a top view of a portion of the first set spline 324D with mapping electrode 382a disposed on support member 374a within aperture 380a of plate electrode 372a. The plate electrode 372a can include a plurality of longitudinally spaced-apart apertures, such as aperture 380a, having a mapping electrode, such as mapping electrode 382a disposed in each aperture for a plurality of mapping electrodes longitudinally spaced apart along the length of the spline 324D for each spine 312D in the basket.

FIG. 3D also illustrates the plate electrode 372a associated with the first set spine 324D includes a surface 376a facing the deflection direction F and a surface 376b facing cavity C that is coupled to support member 374a. The plate electrode 372b associated with second set spline 326D includes a surface 376c facing cavity C that is coupled to support member 374b and a surface 376d facing opposite direction B. An insulator 330D is disposed on the second set 326D of the plurality of splines 312D so as to insulate the associated plate electrode 372b. For example, the insulator 330D is disposed on the second set 326D of the plurality of splines 312D on a side opposite the deflection direction F, which provides for the insulator 330D on surface 376d of the plate electrode 372b. The insulator 330D is not is disposed on the associated electrodes 314D of the first set 324D of the plurality of electrodes. Additionally, the insulator 330D is not disposed on the mapping electrodes, such as mapping electrode 382b.

FIG. 3E illustrates an example electrode assembly 310E in the expanded state and includes a first set 324E of a plurality of splines 312E proximal the deflection direction F and a second set 326E of the plurality of splines 312E distal the deflection direction F. One spline from each set 324E, 326E is illustrated in the example, but the first set 324E can include one or more splines, and the second set 326E can include one or more splines. In the example, a plate electrode 396 is associated with each spline 312E. For instance, each spine 312E is formed from a longitudinally extending plate electrode, which can be used for ablation, such as plate electrode 396a associated with the first set spine 324B and plate electrode 396b associated with the second set spline 326E. The plate electrode 396a associated with the first set spine 324E includes a surface 398a facing the deflection direction F and a surface 398b facing the cavity C. The plate electrode 396b associated with second set spline 326E includes a surface 398c facing the cavity C and a surface 398d facing opposite direction B. An insulator 330E is disposed on the second set 326E of the plurality of splines 312E so as to insulate the associated plate electrode 396b. For example, the insulator 330E is disposed on the second set 326E of the plurality of splines 312E on a side opposite the deflection direction F, which provides for the insulator 330E on surface 398d of the plate electrode 396b. The insulator 330E is also disposed on the associated electrodes 314E of the first set 324E of the plurality of electrodes, such as surface 398b of plate electrode 396a.

In constructing the electroporation catheter 200 of FIG. 2, an elongated shaft 202 having a distal region 204 and a deflection plane 206 is provided. The elongate shaft 202 defines a longitudinal axis A. The elongate shaft 202 includes a lead conductor. An electrode assembly 210 is operably coupled to the distal region 204 and configured to generate an electric field. The electrode assembly 210 is constructed to include a plurality of splines 212, and each spline of the plurality of spline 212 supports an associated electrode 214. In one example, each spline supports an associated ablation electrode, and each ablation electrode is electrically coupled to the lead conductor so as to receive the same electrical signal. The splines 212 are transitionable between a collapsed state and an expanded state. A deflector 220 is constructed or configured in the distal region 204. The deflector 220 is actuable in the expanded state to deflect the electrode assembly 210 off axis A toward a deflection direction F in the deflection plane 206. For example, the deflector 220 is coupled to the shaft 202, and the axis A is determined proximal the deflector 220. The electrode assembly 210 in the expanded state includes a first set 224 of the plurality of splines 212 proximal the deflection direction F and a second set 226 of the plurality of splines 212 distal the deflection direction F. An insulator 230 is disposed, such as deposited, on the second set 226 of the plurality of splines 212 so as to insulate the associated electrode 214. For example, the insulator 230 is deposited on the second set 226 of the plurality of splines 212 on a side opposite the deflection direction F. The insulator 230 not is deposited on the associated electrodes 214 of the first set 224 of the plurality of electrodes.

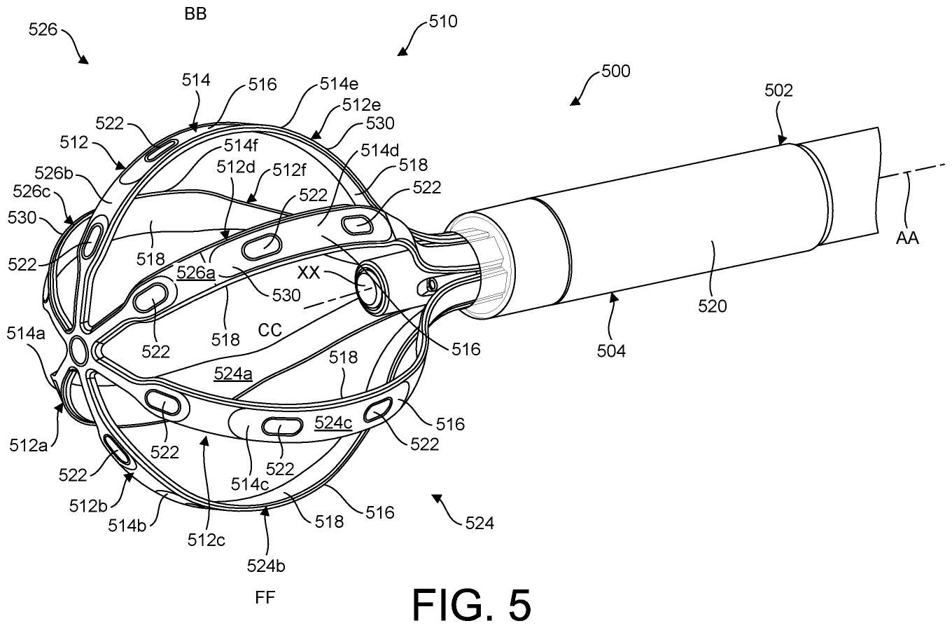

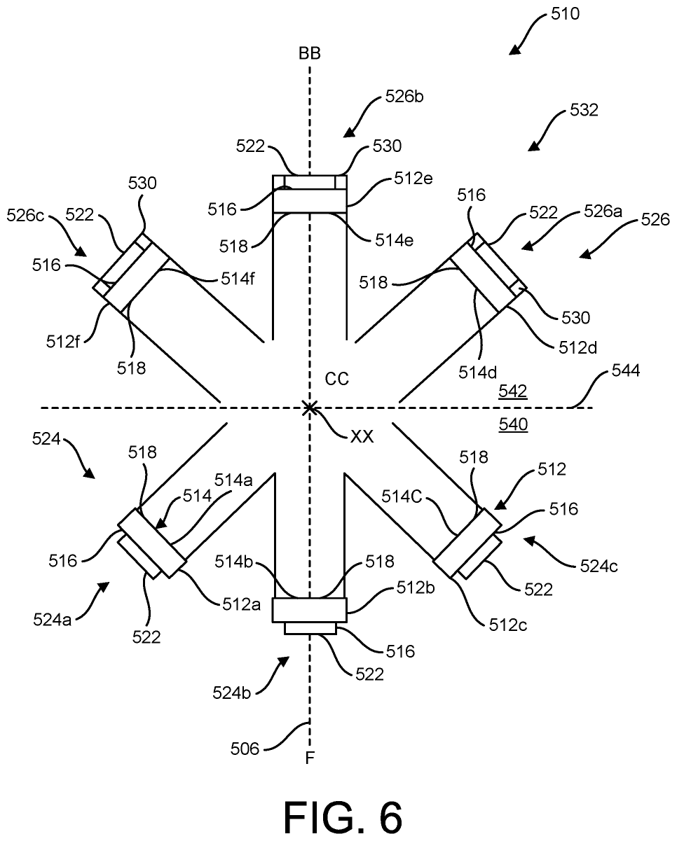

FIG. 5 illustrates a portion of an example electroporation catheter 500 for use with the electrophysiology system 50 and constructed in accordance with the electroporation catheter 200. The example electroporation catheter 500 includes an elongated, tubular shaft 502 having a distal region 504 in a deflection plane 506 (illustrated in FIG. 6). The distal region 504 is configured to be deployed proximate target tissue, such as within a chamber of the patient's heart. The elongate shaft 502 defines a longitudinal axis AA. The longitudinal axis AA is presented as a line passing through a centroid of a cross section of the shaft 502. An electrode assembly 510 is operably coupled to the distal region 504 and configured to generate an electric field. In one example, the electric field effects an irreversible electroporation in selected cardiac tissue. The electrode assembly 510 includes a plurality of ablation electrodes 514 (514a, 514b, 514c, 514d, 514e, 514f, as illustrated) configured as plurality of splines 512 (512a, 512b, 512c, 512d, 512e, 512f, as illustrated). Each ablation electrode 514 includes an outer conductive surface 516 and an opposite, inner conductive surface 518. The inner conductive surfaces 518 face toward cavity CC, and the outer conductive surfaces 516 face away from the cavity CC. The ablation electrodes 514 are configured to receive pulsed electrical signals or waveforms from the electroporation console 130 and create pulsed electric fields sufficient to ablate target tissue via irreversible electroporation. Each spline 512 supports an associated mapping electrode 522. For example, a plurality of associated mapping electrodes 522 can be attached to and longitudinally spaced-apart along each spine 512, such as on the outer conductive surface 516 of each ablation electrode 514. An insulator can be disposed between the mapping electrodes 522 and the associated ablation electrode 514. The mapping electrodes 522 are configured for, among other things, sensing cardiac electrical signals, localization of the electrode assembly 510 within the patient anatomy such as via the EAM system 70, and to determine proximity to target tissue within the anatomy. The mapping electrodes 522 in the electrode assembly 510 can be electrically coupled to a one or more lead conductors that extends the length of the shaft 502 that are configured to carry an electrical signal received at the mapping electrode.

The splines 512 are transitionable between a collapsed state and an expanded state. A deflector 520 is in the distal region 504. The deflector 520 is actuable in the expanded state to deflect the electrode assembly 510 off axis AA toward a deflection direction FF in the deflection plane 506. For example, the deflector 520 is coupled to the shaft 502, and the axis AA is determined proximal the deflector 520. The electrode assembly 510 in the expanded state includes a first set 524 of the plurality of ablation electrode 514 proximal the deflection direction FF and a second set 526 of the plurality of ablation electrodes distal the deflection direction FF. For example, the first set 524 can include three ablation electrodes, 524a, 524b, 524c, one on each of spline 512a, 512b, 512c. In the example, the second set 526 can include three ablation electrodes, 526a, 526b, 526c, one on each of spline 512d, 512e, 512f as illustrated, but other configurations, such as other than six splines 512 or multiple ablation electrodes per spline are contemplated. An insulator 530 is disposed on the second set 526 of the plurality of ablation electrodes 514 so as to cover the outer conductive surface 516 of the associated ablation electrodes 514 with insulation and leave exposed and free from an insulation cover the associated mapping electrodes 522 on the insulated splines 512d, 512e, 512f. In one example, an insulator 530 is not disposed on the inner conductive surfaces 518 of the second set 526 of ablation electrodes 514. In these examples, the insulator 530 not is disposed on the first set 524 of the ablation electrodes 514. In another example, the insulator 530 is disposed on the inner surface 518 of each of the first set 524 of ablation electrodes 514.

In the illustrated example, the splines 512 form a basket 532 coupled to the shaft 502. The basket 532 in the expanded state can include an expanded length, an expanded diameter, and a particular contour. The length of the basket 532 can be determined from the longitudinal distance of the spines 512 from a proximal end to a distal end along a basket axis XX. The diameter of the basket 532 can be determined from the greatest distance between opposite splines 512 in a direction generally perpendicular to the basket axis XX. In the expanded state, the plurality of splines 512 are spaced-apart from each other and form a cavity CC within the basket 532. In the example, the plurality of splines 512 are constructed from a conductive, flexible and resilient material such that the catheter 500 can also be transitionable to a collapsed state. In one example, the catheter 500 is placed in the collapsed state while a deployment sheath is disposed over the splines 512, and the catheter 500 is in a non-operational mode. If the basket 532 is extended from the deployment sheath, or the deployment sheath is retracted from the basket 532, the basket can assume the expanded state and be deployed in an operational mode.

The deflector 520 permits the basket 532 to deflect, or move, off axis AA, such as toward a treatment site in the deflection plane 506. In one example, the deflector 520 allows the basket 532 to pivot or bend basket axis XX in the deflection plane 506 relative to a region on the shaft proximal to the deflector 520. As the basket 532 moves off axis, the basket 532 moves in a deflection direction FF, such as toward the treatment site. As the deflected basket 532 moves toward the axis AA, the basket returns to the axis AA in an opposite direction BB. When the basket 532 in on axis, axis AA and basket axis XX are parallel or colinear.

The electrode assembly 510 in the expanded state includes the first set 524 of ablation electrodes 514 of the plurality of splines 512 proximal the deflection direction FF and the second set 526 of ablation electrodes 514 of the plurality of splines 512 distal the deflection direction FF. In one example, the basket axis XX lies in bisector plane 544 perpendicular to the deflection plane 506. The first set 524 of the ablation electrodes 514 of the plurality of splines 512 are on a proximal side 540 of the bisector plane 534 proximal the deflection direction FF. The second set 526 of the plurality of electrodes 514 of the plurality of splines 512 are on a distal side 542 of the bisector plane 544 distal the deflection direction FF (illustrated in FIG. 6).

In one example, the catheter 500 is be operated in a monopolar mode to generate an electric field for electroporation. For example, the ablation electrodes 514 in the electrode assembly 510 can be electrically coupled to a single lead conductor that extends the length of the shaft 502, or to a set of lead conductors in the shaft 502 that are configured to carry the same electrical signal to generate the electric field. The ablation electrodes 514 on the electrode assembly 510 are configured as one of the anode or the cathode. None of the electrodes in the electrode assembly 510 are configured as the other of the cathode or the anode. Instead, the other of the cathode or the anode is provided in the form of a pad dispersive electrode located on the patient during electroporation. The electrical field is formed between the activated ablation electrodes 514 of the electrode assembly and the pad dispersive electrode. Via the application of the insulator 530 on the second set 526 of the ablation electrodes 514 of the plurality of splines 512, the electrode assembly 510 projects an asymmetrical electric field that focuses electroporation energy into the cavity CC and toward to the deflection direction FF rather than symmetrically in all directions radially from the basket axis XX. Lateral electric field projection corresponds with the deflection plane 506, and the electric field is steered toward the treatment tissue.

FIG. 6 illustrates a front sectioned view of the basket 532 of catheter 500 in the expanded state. The electrode assembly 510 in the expanded state includes the first set 524 of ablation electrodes 514 of the plurality of splines 212 proximal the deflection direction FF and the second set 526 of ablation electrodes 514 of the plurality of splines 512 distal the deflection direction FF. In one example, the basket axis XX lies in bisector plane 534 perpendicular to the deflection plane 506. The first set 524 of the ablation electrodes 514 of the plurality of splines 512 are on a proximal side 540 of the bisector plane 534 proximal the deflection direction FF. The second set 526 of the plurality of electrodes 514 of the plurality of splines 512 are on a distal side 542 of the bisector plane 544 distal the deflection direction FF. The first set 524 includes three ablation electrodes, 524a, 524b, 524c, one on each of spline 512a, 512b, 512c. The second set 526 includes three ablation electrodes, 526a, 526b, 526c, one on each of spline 512d, 512e, 512f as illustrated, but other configurations, such as other than six splines 512 or multiple ablation electrodes per spline are contemplated. An insulator 530 is disposed on the second set 526 of the plurality of ablation electrodes 514 so as to cover the outer conductive surface 516 of the associated ablation electrodes 514 with insulation and leave exposed and free from an insulation cover the associated mapping electrodes 522 on the insulated splines 512d, 512e, 512f. In one example, an insulator 530 is not disposed on the inner conductive surfaces 518 of the second set 526 of ablation electrodes 514. In these examples, the insulator 530 not is disposed on the first set 524 of the ablation electrodes 514. In another example, the insulator 530 is disposed on the inner surface 518 of each of the first set 524 of ablation electrodes 514.

Various modifications and additions can be made to the exemplary embodiments discussed without departing from the scope of the present disclosure. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An electroporation catheter for ablation of cardiac tissue, the electroporation catheter comprising:
an elongated shaft having a distal region and a deflection plane, the elongate shaft defining an axis;
an electrode assembly operably coupled to the distal region and configured to generate an electric field, the electrode assembly having a plurality of splines, wherein each spline supports an associated ablation electrode, the splines transitionable between a collapsed state and an expanded state;
a deflector in the distal region, the deflector actuable in the expanded state to deflect the electrode assembly off axis towards a deflection direction in the deflection plane; wherein the electrode assembly in the expanded state includes a first set of the plurality of splines proximal the deflection direction and a second set of the plurality of splines distal the deflection direction; and
an insulator disposed on the second set of splines so as to insulate at least a portion of the associated ablation electrode.

2. The electroporation catheter of claim 1, wherein the plurality of splines form a basket defining a cavity in the expanded state, and the insulator directs the electric field from each ablation electrode associated with the second set of the plurality of splines towards the cavity.

3. The electroporation catheter of claim 2, wherein the basket is steerable toward a treatment site via the deflector.

4. The electroporation catheter of claim 1, wherein the electrode assembly is configured as one of a cathode and an anode to generate the electric field in a monopolar mode.

5. The electroporation catheter of claim 4, wherein the shaft includes a lead conductor, and each of the associated ablation electrodes are electrically coupled to the lead conductor.

6. The electroporation catheter of claim 1, wherein each ablation electrode associated with the second set of splines include a surface facing the deflection direction and a surface facing opposite the deflection direction, wherein the insulator is disposed on the surface facing opposite the deflection direction.

7. The electroporation catheter of claim 6, wherein each ablation electrode associated with each of the splines include a surface facing the deflection direction and a surface facing the deflection direction, wherein the insulator is disposed on the surface facing opposite the deflection direction.

8. The electroporation catheter of claim 1, wherein ablation electrodes associated with the first set of the plurality of splines are exposed.

9. The electroporation catheter of claim 1, wherein each spine further includes a mapping electrode.

10. The electroporation catheter of claim 9, wherein each spline includes a plurality of exposed mapping electrodes disposed along the spline.

11. An electroporation catheter for ablation of cardiac tissue, the electroporation catheter comprising:
an elongated shaft having a distal region and a deflection plane, the elongated shaft defining an axis;
an electrode assembly operably coupled to the distal region and configured to generate an electric field, the electrode assembly having a plurality of elongated ablation electrodes configured as splines, wherein each ablation electrode includes an outer conductive surface and an inner conductive surface, wherein each spline supports a mapping electrode, the splines transitionable between a collapsed state and an expanded state;
a deflector in the distal region, the deflector actuable in the expanded state to deflect the electrode assembly off axis towards a deflection direction in the deflection plane; wherein the electrode assembly in the expanded state includes a first set of the plurality of splines proximal the deflection direction and a second set of the plurality of splines distal the deflection direction; and
an insulator disposed on the second set of the plurality of splines so as to insulate at least a portion of the outer conductive surface and expose the mapping electrode.

12. The electroporation catheter of claim 11, wherein the plurality of splines form a basket defining a cavity in the expanded state, and wherein each inner conductive surface faces towards the cavity.

13. The electroporation catheter of claim 12, wherein the insulator disposed on the second set of the plurality of splines does not insulate the inner conductive surface.

14. The electroporation catheter of claim 13, wherein the insulator is further disposed on the first set of splines so as to insulate the inner conductive surface and not insulate the outer conductive surface of the first set of splines.

15. The electroporation catheter of claim 11, wherein the shaft includes a lead conductor, and each of the ablation electrodes are electrically coupled to the lead conductor.

16. A method for making an electroporation catheter for ablation of cardiac tissue, the method comprising:
providing an elongated shaft having a distal region and a deflection plane, the elongate shaft defining an axis;
coupling an electrode assembly to the distal region, the electrode assembly configured to generate an electric field, the electrode assembly having a plurality of splines, wherein each spline supports an associated ablation electrode, the splines transitionable between a collapsed state and an expanded state;
providing a deflector in the distal region, the deflector actuable in the expanded state to deflect the electrode assembly off axis towards a deflection direction in the deflection plane; wherein the electrode assembly in the expanded state includes a first set of the plurality of splines proximal the deflection direction and a second set of the plurality of splines distal the deflection direction; and
depositing an insulator on the second set of the plurality of splines so as to insulate at least a portion of the associated electrode.

17. The method of claim 16, wherein providing the shaft includes providing a lead conductor in the shaft, and further comprising electrically coupling each of the associated ablation electrodes to the lead conductor.

18. The method of claim 16, and further including forming each of the splines from the associated ablation electrodes.

19. The method of claim 18, and further forming each of the splines to create a basket in the expanded state.

20. The method of claim 16, and further including attaching a plurality of mapping electrodes to each spline, wherein the insulator is not deposited on the mapping electrodes.
